(19) United States
(12) Patent Application Publication
(10) Pub. No.: US 2023/0165526 A1
(43) Pub. Date: Jun. 1, 2023

(54) METHOD AND APPARATUS FOR AMBULATORY ECG NOISE MITIGATION DURING SWEATING OF PATIENT

(71) Applicant: NimbleHeart, Inc., Sunnyvale, CA (US)

(72) Inventor: Sonal Tambe, Saratoga, CA (US)

(73) Assignee: NimbleHeart, Inc., Sunnyvale, CA (US)

(21) Appl. No.: 18/059,376

(22) Filed: Nov. 28, 2022

Related U.S. Application Data
(60) Provisional application No. 63/283,807, filed on Nov. 29, 2021.

(51) Int. Cl. A61B 5/00; A61B 5/25

(52) U.S. Cl. CPC A61B 5/6802; A61B 5/25

(57) ABSTRACT

Apparatus and methods for an electronic device are provided. The electronic device includes one or more electrodes that are configured to receive electric signals from an individual and a water-resistant, non-absorbent, and non-conductive material that at least partially surrounds the one or more electrodes. The electronic device further includes a wearable harness that is configured to securely position the one or more electrodes to the individual when the wearable harness is worn.

### CROSS REFERENCE TO PRIOR APPLICATION

This application claims the benefit of U.S. Provisional Patent Application No. 63/283,807, entitled as “METHOD AND APPARATUS FOR AMBULATORY ECG NOISE MITIGATION DURING SWEATING OF PATIENT”, filed Nov. 29, 2021, which is incorporated by reference in its entirety.

### FIELD OF THE INVENTION

This disclosure relates to the field of ambulatory medical monitoring devices. Specifically, this disclosure relates to portable electrocardiogram (ECG) devices.

### BACKGROUND

An ECG device has electrodes that record electrical signals from the heart. Electrodes for the ECG are placed on the skin at various locations. Multiple electrodes may be placed at various skin surface locations to monitor an individual from various angles. An ambulatory ECG allows a user free movement as the ECG device is monitoring the individual. A harness may be strapped around the patient to lock one or more of the electrodes into place.

When a patient is wearing an ECG device over a long period of time, body sweat can make the material of the harness stretch more as it soaks up sweat. Sweat around the electrode changes the impedance of electrodes against the skin generating noise in the ECG recoding. There is a need in the art for an ambulatory ECG device that mitigates the effects of long-term use such as sweating.

### SUMMARY

Methods and apparatus are provided for an ambulatory ECG device. An exemplary embodiment is an electronic device. The electronic device includes one or more electrodes that are configured to receive electric signals from an individual and a water-resistant non-conductive material that at least partially surrounds the one or more electrodes. The electronic device further includes a wearable harness that is configured to securely position the one or more electrodes to the individual when the wearable harness is worn. The water-resistant non-conductive material may remain non-conductive when it is wet. The wearable harness may further include one or more sweat patches that each include a material that resists stretching of the wearable harness in a direction that is aligned with the length of the wearable harness. The one or more sweat patches may stretch in one direction where the one or more sweat patches are oriented to stretch in a direction that is perpendicular to the length of the wearable harness to resist stretch of the wearable harness at a position of the sweat patch. The one or more sweat patches may be oriented to stretch only in a direction that is perpendicular to the length of the wearable harness to control stretch of the wearable harness at a position of the sweat patch. The one or more electrodes may not overlap with the one or more sweat patches. The one or more electrodes may have between about 0.25 inches to-about 1 inch of clearance from the one or more sweat patches. The one or more electrodes may have at least about 0.25 inches of clearance from the one or more sweat patches. The one or more sweat patches may include at least one full harness-width patch for tight control and at least one, half harness width patch for moderate control. Each of the one or more sweat patches may be positioned at portions of the wearable harness that are likely to get exposed to sweat from the individual.

Another general aspect is a method for building a harness for receiving electrical signals. The method includes attaching one or more electrodes along a length of a wearable harness that is configured to securely position one or more electrodes to an individual when the wearable harness is worn by the individual, at positions where the electrodes may come into contact with the individual. Each of the one or more electrodes are surrounded by a water-resistant non-conductive material. The water-resistant non-conductive material may remain non-conductive when it is wet. The method may further include attaching one or more patches to the wearable harness to control stretching of the wearable harness. Each of the one or more patches may include a material that resists stretching of the wearable harness in a direction that is aligned with the length of the wearable harness. The one or more patches may include a material that stretches in one direction. Attaching one or more patches may orient the one or more patches to stretch in a direction that is perpendicular to the length of the wearable harness to control stretch of the wearable harness at a position of the patch. The one or more electrodes may not overlap with the one or more patches. The one or more electrodes may have at least about 0.25 inches of clearance from the one or more sweat patches. The one or more electrodes may have between about 0.25 inches to about 1 inch of clearance from the one or more sweat patches. Each of the one or more sweat patches may be positioned at portions of the wearable harness that are likely to receive sweat from the individual.

An exemplary embodiment is an electronic device. The electronic device includes a wearable harness that is configured to wrap around a torso of an individual, one or more electrodes attached to the wearable harness, and one or more sweat patches attached to the wearable harness. The one or more sweat patches may be positioned at positions of the wearable harness that are likely to get exposed to sweat from the individual. The one or more electrodes may each be surrounded by a water-resistant and insulating material that remains non-conductive when it is wet. The water-resistant and insulating material may be a non-conductive rubber. The water-resistant and insulating material may be selected from the group consisting of silicone and a plastic.

### BRIEF DESCRIPTION OF THE DRAWINGS

FIG. 1 is an illustration of an embodiment of a wearable harness.

FIG. 2 is an illustration of an individual wearing an embodiment of the ECG assembly.

FIG. 3 is an illustration of an embodiment of a rubber island assembly without an electrode.

FIG. 4 is an illustration of an embodiment of the electrode surrounded by water-resistant non-conductive material.

FIG. 5 is an illustration of an embodiment of a backing for an electrode that may contact a skin surface of an individual.

### DETAILED DESCRIPTION

The disclosed subject matter is a method and apparatus for an ambulatory electrocardiogram (ECG) assembly that includes electrodes that measure an electrical signal from the heart of an individual. The electrodes are typically placed on a skin surface of an individual. Multiple electrodes may be placed at different parts of a skin surface to simultaneously measure the heart's electrical signal from different angles. The one or more electrodes are connected to a central acquisition unit that captures and records the ECG signals from the electrodes.

The electrodes of the ECG assembly are held against the skin surface of the individual via a wearable harness that wraps around the individual. In an exemplary embodiment, the wearable harness includes a first strap that wraps around the torso just below the chest. Additional one or more-straps go over the shoulders of the individual and attach to the first strap in the front and back of the individual.

Individuals with cardiac risk may need to wear ambulatory ECG devices over long periods of time and/or during monitored Exercise programs such as Cardiac Rehabilitation. The long periods of movement and perspiration caused during those movements may cause ambulatory ECG device to shift position on the body. For example, the one of the straps may be stretchable. The exposure of the individual's sweat to the one of the straps may change the stretchability of the strap and result in one or more electrodes shifting position on the individual's skin. Further, sweat percolating between one or more electrodes and the individual's skin may change input impedance of the ECG device, resulting in noisy ECG signal. Excessive sweating may cause the strap to stretch so much that it may slide on the patient's skin where the electrodes cannot maintain sufficient contact with the patient's skin to capture the ECG signal.

The construction of the disclosed ambulatory ECG assembly minimizes stretch of the wearable harness during sweating. A water-resistant material is used to stitch the wearable harness. Further, water-resistant stretch-controlled tabs are inserted and stitched to the wearable harness in areas where the individual is most likely to sweat. In an exemplary embodiment, the three electrodes are embedded into the first strap. The ECG acquisition unit is positioned on the first strap on the ambulatory ECG assembly in the front of the individual wearing harness. One electrode is to the left of the ECG acquisition unit and one electrode is to the right of the ECG acquisition unit. A third electrode is positioned to on further right of the right electrode to sit on the back on the individual. A first sweat patch is inserted to the left of the left electrode. Similarly, a second sweat patch is inserted to the right of the right electrode. And a third sweat patch is inserted to the right of the third electrode.

In various embodiments, the first strap is stretchable in one direction that is aligned with the length of the first strap. The sweat patches are configured to resist stretching in the direction that is aligned with the length of the first strap. In an exemplary embodiment, the sweat patches are also stretchable in one direction, but the stretchable direction of the sweat patches is turned perpendicular to the length of the first strap. In an exemplary embodiment, the sweat patch simply stretches less than the first strap in any direction. Accordingly, the addition of the sweat patches effectively controls stretch of the first strap. The sweat patches are inserted in most areas that do not include the electrodes or the ECG acquisition unit. As such, the first strap provides stretch control in areas that surround the electrodes while allowing stretch directly at the positions of the electrodes.

To further control the potential loosening from sweating the stretchable parts of the first strap of the wearable harness are stitched together at the edges as well as in the middle portion of the strap using two step zig zag stitch and three step zig zag stitch whereas, multi-layered stretch-controlled sweat patches are attached to the first strap with two step or three step zig zag stitch only at the edges.

Each of the electrodes is surrounded by an insulating island between it and the wearable harness. Both the fabric of the first strap and Velcro change their conductivity when they are wet, such as from the sweat of an individual. The insulating island includes a material that insulates the electrode from the change in conductivity when sweat or other liquid soaks the area around the electrode. The insulating island comprises of water-resistant non-conductive material that doesn't become conductive when wet. Thus, sweat or other liquids that could potentially change the impedance of the ECG device can't affect the performance of the device by generating noise. In an exemplary embodiment, each of the one or more electrode snaps include a socket attached to the insulating island supported on the edges by and Velcro. The sockets snap into the electrode that make contact with the individual's skin for acquiring ECG signal.

In various embodiments, the water-resistant non-conductive or insulating material that surrounds the socket may comprise a non-conductive rubber such as santoprene. Santoprene is biocompatible and used in various products for wire insulation. A sheet of santoprene rubber may be used to isolate the electrodes from the Velcro and fabric of the first strap. A one inch square of santoprene rubber is stitched to the first strap. To the square of rubber, a one inch square of Velcro with a five eighths inch diameter center hole is stitched. The result is a circle of santoprene rubber island surrounded by Velcro. The electrode socket for the electrode is attached in the middle of the santoprene rubber island.

An example of the material that is both nonconductive and water resistant is silicone. In various embodiments, other materials that are biocompatible and do not significantly change conductivity when wet (such as by absorbing water or sweat) may be used. In an exemplary embodiment, the nonconductive water-resistant material may be a plastic. Various plastics that may be used and the disclosed subject matter include, but are not limited to polycarbonate, polypropylene, and polyethylene.

In an exemplary embodiment, the contact portion of the electrodes may include a dry contact solid metal such as Gold-plated brass or stainless steel. Surrounding the dry contact solid metal may be an annular ring type silicone donut. The contact portion of the electrode may be held in place by the standard ECG-snap on rivet and the Velcro that surrounds the santoprene rubber island.

The first strap is stretch controlled at most positions around the first strap except for the area around the electrode. In various embodiments, each electrode has about a one inch area of the harness on either sides that is not stretch controlled. In an exemplary embodiment, each electrode has a one quarter inch area of the harness on all sides that is not stretch controlled.

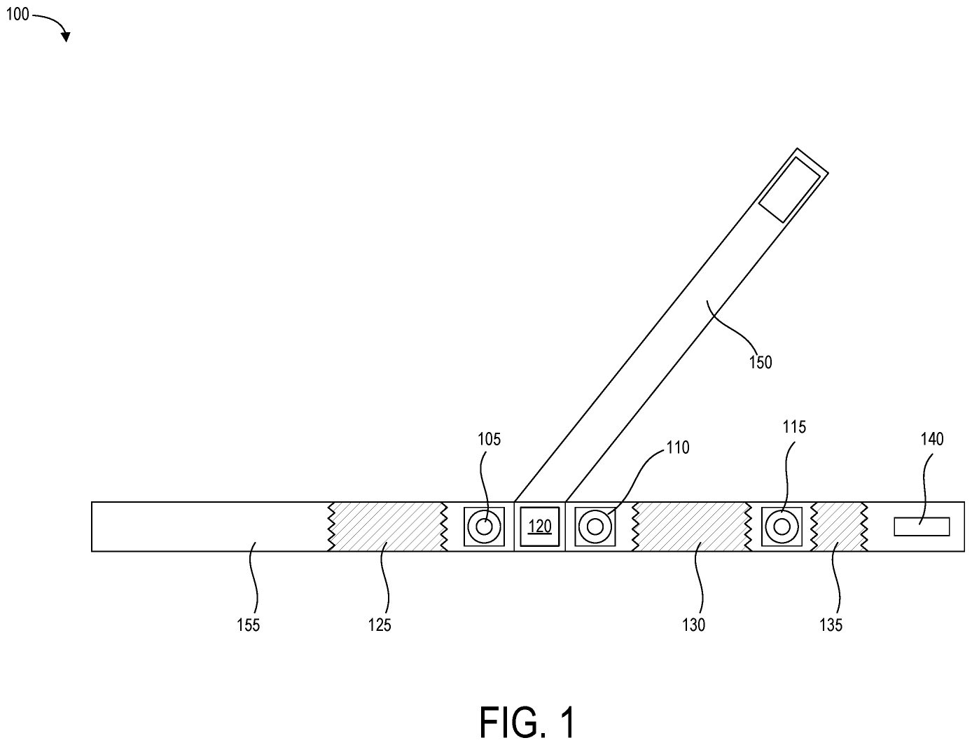

Referring to FIG. 1, FIG. 1 is an illustration of an embodiment of an inside portion of a wearable harness 100. The wearable harness 100 holds the various components of the ambulatory ECG assembly to the body of an individual such that the individual may move around freely. Ambulatory ECGs are used on cardiac risk individuals to monitor the heart over periods of time. The individual may sweat profusely under certain circumstances, which may affect the signals that are collected by the ambulatory ECG assembly and cause noise. The disclosed wearable harness 100 mitigates the noise that is produced by sweat, which may be produced by an individual undergoing strenuous activity.

The wearable harness 100 includes one or more electrodes that are configured to measure electrical signals from the heart of an individual that is wearing the wearable harness 100. In the exemplary embodiment that is shown in FIG. 1, the wearable harness includes three electrodes. A first electrode 105 is positioned on the wearable harness 100 to the left of a ECG acquisition unit 120. A second electrode 110 is positioned opposite the first electrode 105 on the right of the ECG acquisition unit 120. A third electrode 115 is positioned further to the right on the wearable harness 100.

When the wearable harness 100 is worn by an individual, the first electrode 105 may be positioned on the chest of the individual to the left of the heart. The second electrode 110 may be positioned on the chest of the individual to the right of the heart. The third electrode 115 may be positioned on the right side of the individual on the rib cage. Various embodiments may position the one or more electrodes at other locations of the wearable harness 100. For example, one or more electrodes may be positioned on the shoulder strap 150.

Features on the wearable harness 100 that mitigate noise that is caused by excessive sweat include one or more sweat patches and insulating islands between the one or more electrodes and the fabric of the wearable harness 100. The term “insulating,” when used herein, refers to high resistance or otherwise non-conductive materials that tend to resist the flow of electric charge. In an exemplary embodiment, the term nonconductive, as used herein, refers to materials that have a surface conductivity of greater than 20 MS2 per square. The terms “nonconductive” and “insulating”, as used herein, are used interchangeably and have the same meaning. The term “water resistant,” when used herein, refers to materials that do not absorb water and/or change their conductivity when exposed to water. In an exemplary embodiment, the term water-resistant, as used herein, refers to materials that don't change their conductivity by water/sweat absorption. An example of a water-resistant and insulting material that may comprise the insulating island is rubber.

The one or more sweat patches, which are indicated by the shaded portions on the wearable harness 100, control stretchability of the wearable harness 100 at the positions of the sweat patches. The first strap 155 of the wearable harness 100 is configured to wrap around the torso of an individual such that the first electrode 105 and the second electrode 110 are positioned on either side of the heart. Thus, the electrodes measure a heart signal from different angles closest to the heart.

The first strap 155 may comprise a stretchable fabric that stretches more in one direction than the other direction. Fabrics that stretch more in one direction than other directions are referred herein as stretchable in one direction even where the fabric is still stretchable in the other directions to a lesser degree. The first strap 155 is stretchable in one direction that is oriented along the length of the first strap 155. Thus, the length, but not the width of the first strap 155 may elongate as force is applied to the first strap 155.

The first strap 155 may comprise two layers of fabric: an inside layer that touches the skin of the individual that is wearing the wearable harness 100, and an outside layer that faces away from the individual that is wearing the wearable harness 100. Wiring for the electrodes and sweat patches may be inserted inside the two layers of the first strap 155. All components that are inside the first strap 155 may be reasonably protected from sweat or other liquids where the first strap 155 is made from water-resistant fabric. The one or more electrodes may snap into a rivet, be designed to be water-tight, and be oriented on the inside portion of the first strap 155. In an exemplary embodiment, a strip of rubber is stitched to a circular hole on the fabric at the position of the electrode. To the rubber, a small hole is made and surrounds an electrode socket that is electrically connected to the ECG acquisition unit 120.

The one or more sweat patches may comprise one or more layers of fabric that is stretchable in one direction. In various embodiments, the one or more layers of fabric that comprise the sweat patches may be a laminated fabric. In various embodiments, the one or more layers of fabric may be the same material that comprises the first strap. The one or more layers of fabric may be attached to one another via a three step zig zag stitch. In various embodiments, the entire sweat patch is inserted inside the first strap 155 such that the first strap 155 covers each sweat patch. Thus, the three step zig zag stitch, which fastens the one or more layers of the sweat patch together, may be covered by the first strap 155 and protected from liquids. Thus, liquids from sweat or other sources such as the elements that do not penetrate the first strap 155 will have minimal effect on the stretchability of the stitching for the sweat patch.

The one or more sweat patches may be attached to the first strap 155 in an orientation such that the stretchable direction of the sweat patches is perpendicular to the length of the first strap. Thus, the sweat patches may effectively limit the stretchability of the first strap in the locations where the sweat patches are attached. By this, the stretchability of the first strap is controlled at those locations. Stretchability is controlled over most of the first strap, but not at the positions of the electrodes.

The sweat patches may be attached to the first strap 155 at various positions. In the embodiment shown in FIG. 1, a first sweat patch 125 is attached to the first strap 155 to the left of the first electrode 105. A second sweat patch 130 is attached to the first strap 155 to the right of the second electrode 110. A third sweat patch 135 is attached to the first strap 155 to the right of the third electrode 115. The stretchability of the first strap 155 is controlled in locations of the sweat patches and is free to stretch at other locations.

In various embodiments, the sweat patches may be reduced in width to offer moderate stretch control. For example, a sweat patch that has a width that is half a width of the first strap 155 may be attached to the first strap 155 to provide moderate stretch control that allows for more stretchability than a sweat patch with a full width of the first strap 155 that is attached to the first strap 155.

In an exemplary embodiment, the first sweat patch 125 has a full width of the first strap 155 and provide maximum stretch control. The second sweat patch 130, which is positioned between the second electrode 110 and the third electrode 115, comprises a width that is half the width of the first strap 155. The third sweat patch, like the first sweat patch, has a width that is at least the full width of the first strap 155. Accordingly, the sweat patches provide maximum sweat control to the left of the first electrode 105 and to the right of the third electrode 115. The second sweat patch 130 provides moderate stretch control between the second electrode 110 and the third electrode 115.

The shoulder strap 150, as shown in FIG. 1, does not include electrodes, and is therefore configured not to be stretchable. Nor, for that reason, does the shoulder strap 150 include sweat patches. In an exemplary embodiment, the shoulder strap 150 comprises two layers. One layer is a stretchable material, and the other layer is a non-stretchable material, which effectively renders the shoulder strap 150 non-stretchable.

In various embodiments where the shoulder strap 150 includes one or more electrodes, the shoulder strap 150 may be stretchable in one direction like the first strap. Likewise, in cases where the shoulder strap 150 includes one or more electrodes, the shoulder strap 150 may include one or more sweat patches to control stretchability at locations away from the electrodes.

The ECG acquisition unit 120 powers and receives signals from the one or more electrodes. In an exemplary embodiment, the ECG acquisition unit 120 includes a battery an Amplifier, an Analog to digital convertor, and a processor coupled to a memory. The battery may be capable of powering the electrodes and the ECG acquisition unit 120. The processor in the ECG acquisition unit may be an integrated circuit such as a central processing unit (CPU), a graphics processing unit (GPU), a complex programmable logic device (CPLD), and field programmable gate array (FPGA) and an application specific integrated circuit (ASIC). In various embodiments, the ECG acquisition unit 120 may be delocalized or may be a cloud compute system.

The one or more electrodes are protected from noise-causing effects of sweat or other liquid via an insulating island. The one or more electrodes are electrically separated from the fabric of the first strap 155 and are instead surrounded by a strip of non-conductive rubber. In various embodiments, the non-conductive rubber comprises santoprene. Other non-conductive materials may similarly be used to insulate the electrode from the fabric of the first strap 155.

In an exemplary embodiment, the electrodes may include an electrode socket that is connected to wires that are electrically connected to the ECG acquisition unit 120. To the electrode socket, a contact portion of the electrode may be snapped into position. The contact portion of each electrode may comprise a conductive metal surface in the middle that is surrounded by a circular silicone donut that immobilizes the contact portion to a skin surface of the individual. The size of the silicone donut may vary. In an exemplary embodiment, the diameter of the conductive metal electrode is about 0.75 inches. The diameter of the circular donut that surrounds the conductive metal is about 1.5 inches. In various embodiments, the diameter of the circular donut may be increased to account for more sweat or increased movement of the individual. For example, the diameter of the circular silicone donut may be about 2 inches.

In addition to the electrode socket, a layer of Velcro may act to fasten the contact portion of the electrode to the first strap 155. However, because Velcro may absorb sweat and is partially conductive, the Velcro does not contact the metal part of the electrode that conducts the electrical signals. Instead, a hole is cut from a strip of Velcro whereby the Velcro only contacts the outside nonconductive parts of the contact portion such as the silicone donut. In the middle of the Velcro hole is an insulating material that contacts the electrode. In various embodiments, the insulating material is rubber. In one example, a layer of santoprene separates the conductive part of each electrode from the fabric of the first strap 155.

The wearable harness 100 may include one or more fastening elements that secure the straps of the wearable harness 100 to the individual. In various embodiments, strips of Velcro are used as the fastening elements. For instance, the strip of Velcro 140, which is on the inside portion of the wearable harness 100 on the right side of the first strap 155, may be secured to another strip of Velcro, such as a strip of Velcro on the outside portion of the wearable harness 100 on the left side of the first strap 155. As strips of Velcro may typically not be stretchable, the portions of the wearable harness 100 that are attached to Velcro may resist stretching in any direction.

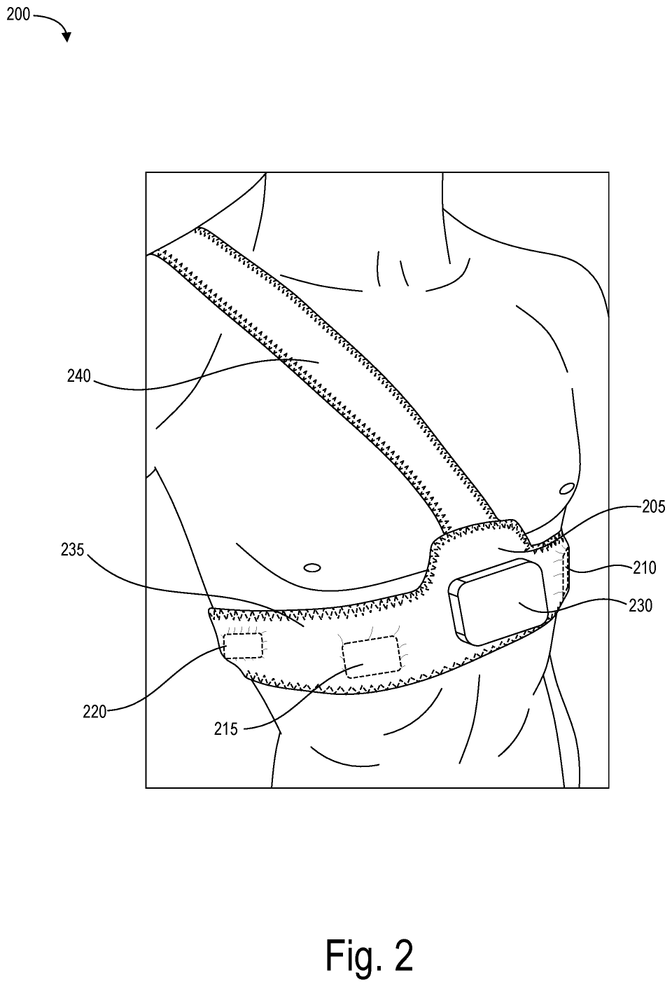

Referring to FIG. 2, FIG. 2 is an illustration of an individual wearing an embodiment of the ECG assembly 200. The ECG assembly 200 includes a wearable harness 205 with a first strap 235 and a shoulder strap 240, a ECG acquisition unit 230, and one or more electrodes. The exemplary embodiment shown in FIG. 2 includes three electrodes that are advantageously placed to efficiently receive electrical heart signals from the most valuable angles.

Accordingly, a first electrode 210 is placed on a skin surface below the chest to the left of the sternum. A second electrode 215 is placed on a skin surface below the chest to the right of the sternum. And a third electrode 220 is placed on a skin surface on the right half of the back Each of the electrodes shown in FIG. 2 is covered at least by the outside fabric layer of the first strap 235.

A contact portion of the electrodes makes contact with the skin surface of the individual. The skin surface of the individual is also in contact with the inner layer of the first strap 235 and an inner layer of the shoulder strap 240. Thus, the first strap 235 and the shoulder strap 240 may be exposed to perspiration or other fluid from the skin of the individual. The stretchability of the first strap 235 and the shoulder strap 240 may change when wet. Further, an impedance of the electrodes may change if they become wet. Both issues may affect the precision of signals that are received by the electrodes.

Firstly, the fabric that stretches may loosen when wet, which may affect a contact quality between the electrode and the skin surface. Secondly, the electrodes may soak up sweat which can change the impedance of the electrode and result in a change in signal. And thirdly, the silicone donuts may not hold up well on the skin due to loosening of the wearable harness and sweat seeping between the silicone donut and the skin surface. The first of these issues is particularly mitigated by the design of the electrode to be insulated from conductive materials.

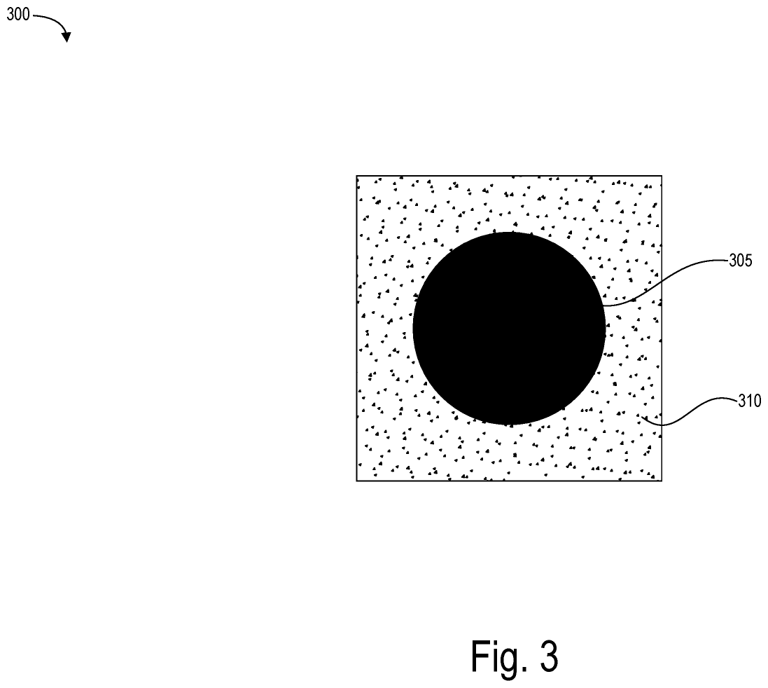

Referring to FIG. 3, FIG. 3 is an illustration of an embodiment of a rubber island assembly 300 without an electrode. The rubber island assembly 300 helps to insulate the one or more electrodes of the ambulatory ECG assembly from conductive materials while securing the electrodes from movement that could affect the input signals received by the electrodes. In an exemplary embodiment, the rubber island assembly 300 may be stitched to an inner layer of the first strap 155 such that the rubber island assembly 300 faces the individual's skin surface as the individual is wearing the ambulatory ECG assembly. A rivet may be attached to the rubber island assembly 300 to facilitate the electrode socket connection traversing through the rubber island assembly 300

The center 305 of the rubber island assembly 300 is a non-conductive material. In an exemplary embodiment, a strip of santoprene rubber comprises the center 305 of the rubber island assembly 300. As santoprene is biocompatible, the individuals wearing the monitor will not develop skin rashes or allergic effects from coming into contact with santoprene. Various embodiments may comprise other non-conductive materials.

Surrounding the center 305 is a Velcro layer 310 or other material that acts to fasten the contact portion of the electrode to the first strap 155. The Velcro layer 310 may absorb fluids and change in conductivity when the individual sweats profusely such as when the individual engages in vigorous exercise. As such, the Velcro layer 310 is insulated from the center 305, which is where the electrode protrudes from the fabric of the wearable harness 100.

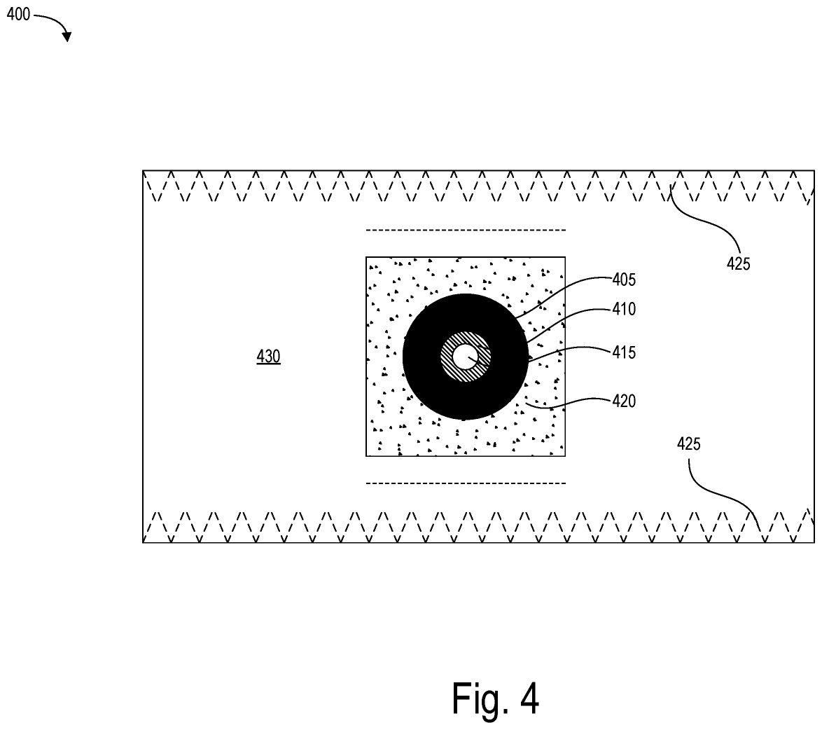

Referring to FIG. 4, FIG. 4 is an illustration 400 of an embodiment of the harness portion of the electrode surrounded by water-resistant material. The harness portion of the electrode is attached to the wearable harness 100 while the contact portion is separatable from the wearable harness 100. The electrode shown in FIG. 4 is a socket for an electrode socket that may connect to a contact portion of the electrode. The electrode is insulated from a potentially conductive Velcro layer 420 by an insulating layer 405. In an exemplary embodiment, the insulating layer 405 comprises santoprene rubber.

The electrode socket may include a metal rim 410 of the socket that surrounds a socket portion 415. The metal rim 410 and a socket portion 415 are electrically connected to each other. A contact portion, which is not shown in FIG. 4, may be snapped into the socket portion 415 to electrically connect the contact portion to the electrode. Further, the contact portion may be additionally secured to the electrode via the Velcro Layer 420. The Velcro layer 420 may change a conductivity when wet, which is why it is separated from the electrode socket by the insulating layer 405. However, even though the Velcro layer 420 should not come into electrical contact with the signal receiving part of the electrode, it may come into contact with the silicone donut without changing the conductivity of the electrode.

The electrode may be inserted into the first strap 155, the shoulder strap 150, or additional straps of embodiments of the ambulatory ECG assembly that include more straps that secure the wearable harness 100 to the individual. In the embodiment shown in FIG. 1, the electrode is attached to the first strap 155 and isolated from the conductive properties of the fabric 430 of the first strap 155. In an exemplary embodiment, the two layers of the first strap 155 are sewn together via three step zig zag stitching 425, which mitigates the stretching between the layers of the first strap 155.

Two step and three step zig zag stitching may be used to sew most of the fabric-to-fabric connections of the wearable harness. For instance, the multiple layers of the sweat patch may be sewn together via three step zig zag stitching, which is not exposed to the outside of the wearable harness. The sweat patch is then sewn to the wearable harness via a two step zig zag stitch. Thus, the stitches that bind the multiple layers of the sweat patch do not get exposed to sweat or other liquids and thus do not suffer any effects such as loosening.

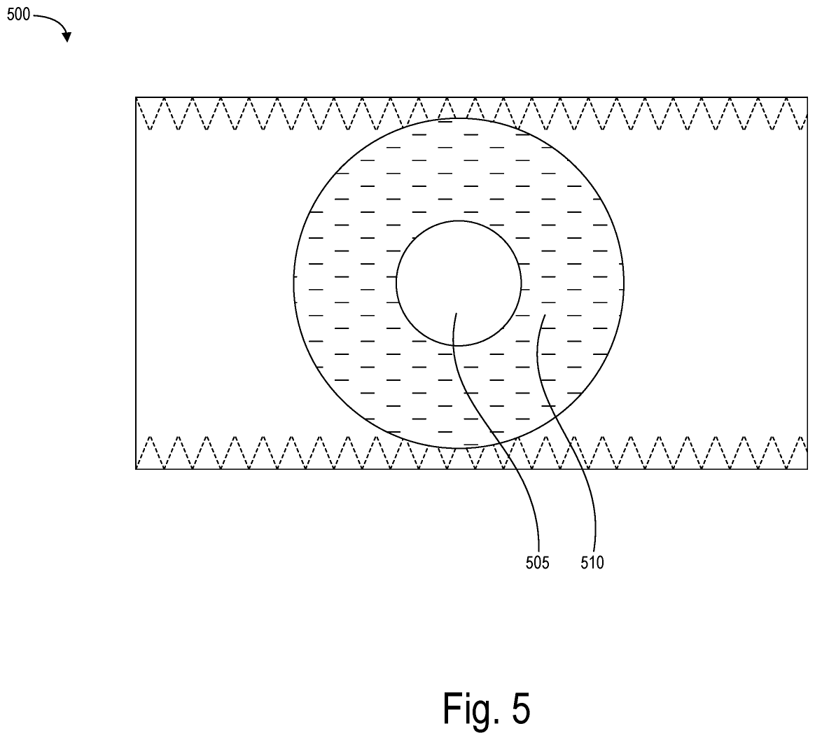

Referring to FIG. 5, FIG. 5 is an illustration 500 of an embodiment of a backing for a contact portion that may contact a skin surface of an individual. The contact portion may include conductive metal surface 505 that is surrounded by a silicone donut 510. The contact portion may be fastened to the harness portion of the electrode in various ways. In an exemplary embodiment, the contact portion is fastened to the harness portion via an electrode socket whereby the contact portion includes a conductive post, which is not shown, that is inserted into a socket portion 415 of the harness portion.

The conductive metal surface 505 may be a variety of shapes and comprise a variety of conductive metals. In an exemplary embodiment, the conductive metal surface 505 comprises brass and is gold plated to increase conductivity. In various embodiments, the conductive metal surface 505 comprises steel. The contact portion may include a spring backing. The silicone donut 510 may help secure the contact portion to the skin surface through a static friction force between the silicone donut 510 and the skin surface. In various embodiments, a material other than silicone may comprise the silicone donut 510.

The size and shape of the silicone donut 510 may vary. In an exemplary embodiment, the silicone donut 510 has a diameter of about two inches. In various embodiments, the silicone donut has a diameter of about 1.5 inches. The back side of the silicone donut 510, which is not shown, may include a layer of Velcro that can fasten the silicone donut to the Velcro layer 420.

Many variations may be made to the embodiments described herein. All variations, including combinations of variations, are intended to be included within the scope of this disclosure. The description of the embodiments herein can be practiced in many ways. Any terminology used herein should not be construed as restricting the features or aspects of the disclosed subject matter. The scope should instead be construed in accordance with the appended claims.

1. An electronic device for receiving electrical signals, the electronic device comprising:
one or more electrodes that are configured to receive electric signals from an individual;
a water-resistant non-conductive material that at least partially surrounds the one or more electrodes; and
a wearable harness that is configured to securely position the one or more electrodes to the individual when the wearable harness is worn.

2. The electronic device of claim 1, wherein the water-resistant non-conductive material remains non-conductive while the water-resistant non-conductive material is wet.

3. The electronic device of claim 1, further comprising one or more sweat patches;
wherein each sweat patch comprises a material that resists stretching of the wearable harness in a direction that is aligned with a length of the wearable harness.

4. The electronic device of claim 3, wherein the one or more sweat patches stretch in one direction; and
wherein the one or more sweat patches are oriented to stretch in a direction that is perpendicular to the length of the wearable harness to resist stretch of the wearable harness at positions of the one or more sweat patches.

5. The electronic device of claim 3 wherein the one or more electrodes do not overlap with the one or more sweat patches.

6. The electronic device of claim 5, wherein the one or more electrodes have at least about 0.25 inches of clearance from the one or more sweat patches.

7. The electronic device of claim 6, wherein the one or more electrodes have between about 0.25 inches to about 1 inch of clearance from the one or more sweat patches.

8. The electronic device of claim 7, wherein the one or more sweat patches comprise at least one full-width patch for tight control and at least one half-width patch for moderate control.

9. A method for building a harness for receiving electrical signals, the method comprising:
attaching one or more electrodes along a length of a wearable harness, that is configured to securely position the one or more electrodes to an individual when the wearable harness is worn by the individual, at positions where the one or more electrodes may come into contact with the individual; and
wherein each of the one or more electrodes are surrounded by a water-resistant non-conductive material.

10. The method of claim 9, wherein the water-resistant non-conductive material remains non-conductive when the water-resistant non-conductive material is wet.

11. The method of claim 10, wherein the wearable harness comprises one or more patches, the one or more patches comprising a material that resists stretching of the wearable harness in a direction that is aligned with the length of the wearable harness.

12. The method of claim 11, wherein the one or more patches comprise a material that stretches in one direction;
wherein the one or more patches are oriented on the wearable harness to stretch in a direction that is perpendicular to the length of the wearable harness to control stretch of the wearable harness at a position of each patch; and
wherein the one or more electrodes do not overlap with the one or more patches.

13. The method of claim 12, wherein the one or more electrodes have at least about 0.25 inches of clearance from the one or more patches.

14. The method of claim 13, wherein the one or more electrodes have between about 0.25 inches to about 1 inch of clearance from the one or more patches.

15. The method of claim 14, wherein each of the one or more patches are positioned at portions of the wearable harness that are likely to receive sweat from the individual.

16. An electronic device for receiving electrical signals, the electronic device comprising:
a wearable harness that is configured to wrap around a torso of an individual;
one or more electrodes attached to the wearable harness; and
one or more sweat patches attached to the wearable harness.

17. The electronic device of claim 16, wherein the one or more sweat patches are positioned at positions of the wearable harness that are likely to get exposed to sweat from the individual.

18. The electronic device of claim 17, wherein the one or more electrodes are each surrounded by a water-resistant and insulating material that remains non-conductive when it is wet.

19. The electronic device of claim 18, wherein the water-resistant and insulating material is a non-conductive rubber.

20. The electronic device of claim 18, wherein the water-resistant and insulating material is selected from the group consisting of silicone and a plastic.
